(19) United States
(12) Patent Application Publication
(10) Pub. No.: US 2026/0026817 A1
(43) Pub. Date: Jan. 29, 2026

(54) WOUND CLOSURE SYSTEM

(71) Applicant: Sterling Innovations, LLC, Scottsdale, AZ (US)

(72) Inventor: Danton S. Dungy, Scottsdale, AZ (US)

(21) Appl. No.: 19/272,377

(22) Filed: Jul. 17, 2025

Related U.S. Application Data
(60) Provisional application No. 63/674,739, filed on Jul. 23, 2024.

(51) Int. Cl. A61B 17/08; A61B 17/3209

(52) U.S. Cl. CPC A61B 17/085; A61B 2017/081; A61B 2017/088; A61B 17/32093

(57) ABSTRACT

A system is provided for closing a wound. The system provides a substrate that attaches to a tissue. For instance, the substrate may be a mesh. The substrate supports a zipper. The zipper may be opened to provide access to the tissue for access to a wound, or for forming an incision. The zipper may be closed to compress the tissue adjacent to the wound or incision in order to close the wound or incision for healing.

### CROSS-REFERENCE TO RELATED APPLICATION

This application claims priority to U.S. Provisional Patent Application No. 63/674,739, filed Jul. 23, 2024, and entitled “WOUND CLOSURE SYSTEM,” the disclosures of which are hereby incorporated by reference herein.

### BACKGROUND

### Field

The various aspects and embodiments described herein relate to wound closure systems, and more specifically, to wound closure systems.

### Description of the Related Art

During medical procedures incisions are often made and need to be closed after the procedure. Moreover, injuries may include cuts that also need to be closed. Conventional closure mechanisms include sutures, tape, glue and the like. However, these mechanisms often result in poor registration of the opposing wound faces to each other, leading to scarring, irregular scarring, and other healing complications. Moreover, some of these mechanisms are prone to infection or inadvertent premature removal. Thus, there remains a need for a wound closure mechanism that improves patient outcomes.

### SUMMARY

A wound closure device is disclosed. The device may have a bonding layer, a closure layer, and a closure securement device. The bonding layer may be adherable to a tissue adjacent a surgical incision site. The closure layer may be lying in a first plane and may be attached to the bonding layer. The closure layer may include a selective joining member configured to be selectively closed and opened to selectively define a passage through the closure layer. The closure securement device may be attached to at least one of the bonding layer and the closure layer to selectively inhibit transition of the selective joining member from a selectively closed configuration to a selectively opened configuration.

The wound closure device may have one or more additional aspects. For instance, the bonding layer may be a structural mesh adherable to the tissue by glue. The selective joining member may be a zipper. The selective joining member may be a zipper having a first element row with a row of teeth and a second element row with a row of teeth disposed adjacent the first element row and selectively engageable to the first element row by a zipper slider. The first element row and the second element row may be disposed on opposite sides of the passage in the selectively opened configuration.

In various instances, the closure securement device includes a slider capture boss defining a slider capture cavity conforming in shape to at least a portion of the selective joining member to retain the selective joining member in a fixed position. In various instances, the closure securement device includes a slider capture boss adjacent to an end of the first element row and defining a slider capture cavity shaped to receive the zipper slider in snap-fittable relation. In various instances, the closure securement device includes a slider closure lid attachable to the slider capture boss to restrict removal of the zipper slider from the slider capture cavity. The first plane may include one or more complex curvature.

In various instances, the bonding layer is lying in a second plane, and the first plane and the second plane are parallel. Moreover, in various instances the passage is through the closure layer and the bonding layer. The passage may be through the closure layer and not through the bonding layer. The passage may be through the closure layer and not through the bonding layer, and the passage may expose the bonding layer for cutting by a cutting instrument introduced through the passage in the selectively opened configuration.

The selective joining member may include a slider puller attached to the zipper slider. The closure securement device may include a slider capture boss defining a slider capture cavity conforming in shape to at least a portion of the slider puller to retain the selective joining member in a fixed position by fixing the slider puller in the slider capture cavity. The at least one slider capture tab may include a boss extending from a floor of the slider capture cavity to penetrate an aperture of the slider puller to restrict translation of the zipper slider along the first element row.

In various instances, the closure layer includes a first tape member supporting the first element row and attached to the structural mesh of the bonding layer. The closure layer may include a second tape member supporting the second element row and attached to the structural mesh of the bonding layer. The bonding layer may be cuttable along a path adjacent to and disposed between both the first tape member and the second tape member to allow selective opening of the passage. The passage may coincide with the path.

In various instances, the closure layer has a first tape member supporting the first element row and attached to the structural mesh of the bonding layer. The closure layer may also have a second tape member supporting the second element row and attached to the structural mesh of the bonding layer. The first element row and the second element row may be selectively engageable to each other to selectively close a passage between the first element row and the second element row.

Another example wound closure device is provided. The device may include a structural mesh gluable to a tissue. The device may include a zipper attached to a surface of the structural mesh in a first direction. The zipper may be selectively openable to provide access to the tissue for cutting. The zipper may be selectively closable to compress the tissue in compression perpendicular to the first direction for healing of an incision at least partially parallel to the first direction.

In some embodiments, the wound closure device has a slider capture boss disposed at an end of the zipper to capture a zipper slider of the zipper to prevent selective opening of the zipper during healing of the incision after the zipper is selectively closed.

A method is provided. The method may be a method of closing a wound with a wound closure device. The method may include gluing a structural mesh to a tissue, the structural mesh having a zipper attached to a surface of the structural mesh in a first direction. The method may include unzipping the zipper along the first direction to provide an opening in the first direction. The method may include cutting the structural mesh through the opening. The method may include cutting the tissue underlying the opening to form an incision. The method may include zipping the zipper along the first direction to close the incision for healing. Further aspects may also be included.

### BRIEF DESCRIPTION OF THE DRAWINGS

Other apparatus, methods, features, and advantages of the present disclosure will be apparent to one skilled in the art upon examination of the following figures and detailed description. Component parts shown in the drawings are not necessarily to scale and may be exaggerated to better illustrate the important features of the present disclosure.

FIGS. 1A-1C illustrates features of a zipper wound closure system, in accordance with various embodiments;

FIGS. 2A-2C illustrate detail views of a closure securement device of a zipper wound closure system, in accordance with various embodiments; and

FIG. 3 illustrates a method of zipper wound closure, in accordance with various embodiments.

### DETAILED DESCRIPTION

During medical procedures incisions are often made and need to be closed after the procedure. Moreover, injuries may include cuts that also need to be closed. Conventional closure mechanisms include sutures, tape, glue and the like. However, these mechanisms often result in poor registration of the opposing wound faces to each other, leading to scarring, irregular scarring, and other healing complications. Moreover, some of these mechanisms are prone to infection or inadvertent premature removal. Thus, there remains a need for a wound closure mechanism that improves patient outcomes.

Classic wound closures have various problems. For instance, staples may leave leaky wounds, with increased infection risk. Wounds closed by staples may need bulky dressings and multiple dressing changes. Wounds closed by staples may cause delays in hygiene, such as showering. Typically, a provider must remove staples, and typically removal is painful. Such wounds often exhibit poor cosmesis, and potentially an unsightly appearance after healing.

In a further example, sutures may have various problems. In some instances, sutures may be too loose, resulting in slower healing and more pronounced scarring. In other instances, sutures may be too tight, resulting in other undesirable effects.

Research has determined that by returning the tissue positioning and alignment as near as possible to the pre-wound arrangement, scarring can be reduced. However, mechanisms such as using markers to aid in visual alignment of tissue are subject to error. Taping and other closures are often time consuming to apply due to the recommended subcuticular closure layer, may be misunderstood as a dressing that is prematurely removed, and may exhibit variable results so that wound cosmesis varies with wide and thin areas of scarring. Yet furthermore, 5-15 minutes of additional wound closure time may be needed such as to facilitate subcuticular closure.

The systems and methods herein facilitate better alignment of tissue cells, minimizing scarring. The system is less time consuming than taping because the subcuticular closure layer may be omitted. The system may improve the variable results with wound cosmesis so that wide and thin areas of scarring are improved and exhibit less variation. The system is less likely to be misunderstood as a dressing that is prematurely removed. The system protects skin edges during surgery for surgical incisions. Moreover, different lengths are available, to accommodate different incisions on different areas of the body.

The systems and methods herein may include applying a wound closure system to a patient's skin prior to skin incision, making an incision while the system is applied to the patient's skin, then using the system to close the incision and to maintain closure of the incision during healing. Moreover, all or part of the system herein may be manufactured with different colors. For instance, all or part of the system may be made in colors approximating a skin tone of a patient, or having artistic designs, such as mimicking a tattoo.

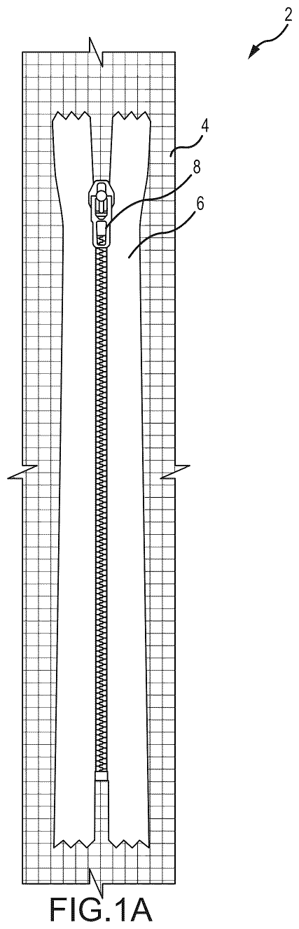

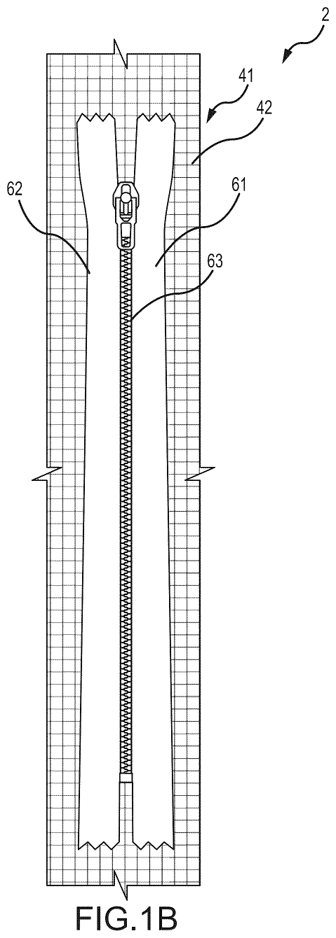

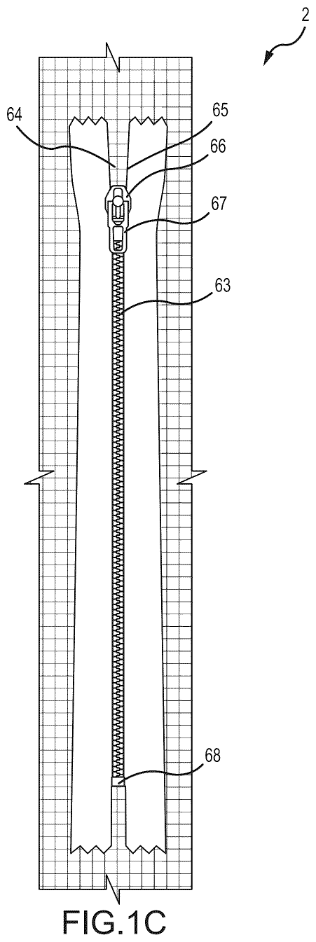

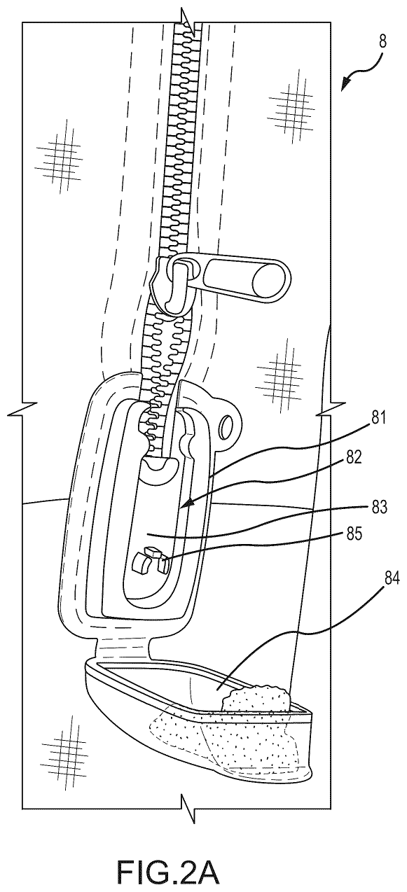

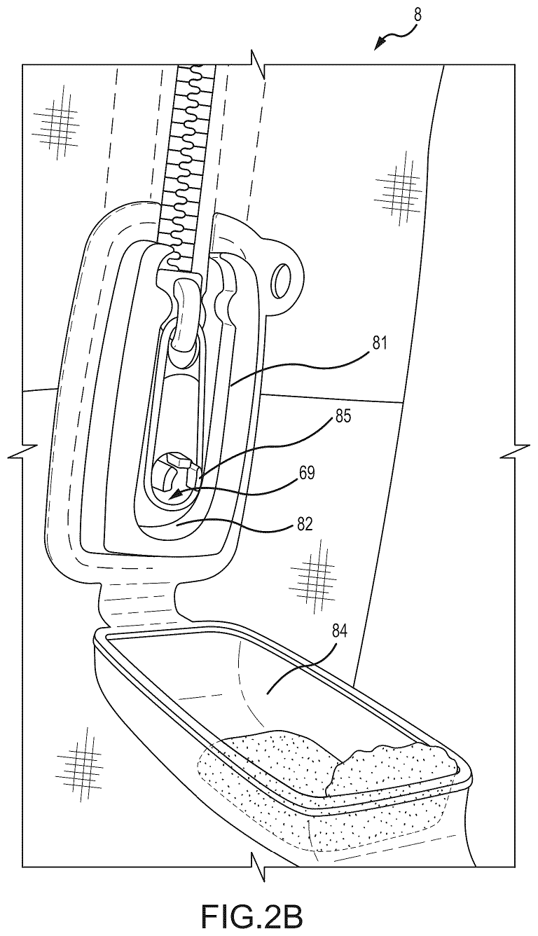

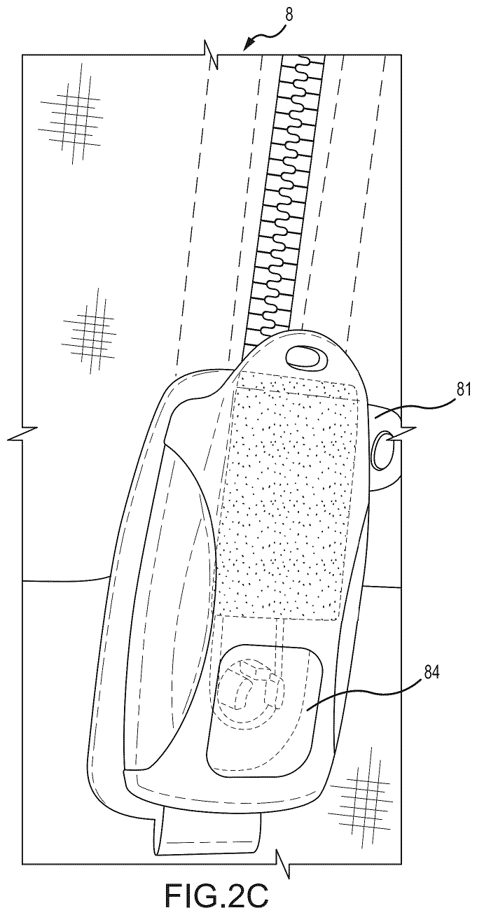

Turning now to FIGS. 1A-IC, a wound closure system 2 is provided. The wound closure system 2 may have a movable closure that selectively opens to allow access to a wound or creating of an incision, and that selectively closes, to close the wound or incision for healing. The wound closure system 2 may include a bonding layer 4. The bonding layer 4 may affix to a tissue, such as to a patient's skin. The wound closure system 2 may include a closure layer 6. The closure layer 6 may be connected to the bonding layer 4 and may selectively open and close, to allow access to a wound or creation of the incision, and then to allow closure of the wound or incision, transferring force through the bonding layer 4 to the tissue to facilitate maintaining the wound or incision in a closed and healing relation. Finally, the wound closure system 2 may have a closure securement device 8 (FIGS. 2A-2C). The closure securement device 8 (FIGS. 2A-2C) may be operable to prevent unwanted opening of the closure layer 6, such as prematurely during healing of the wound or incision. In various embodiments, the bonding layer 4 may advantageously distribute topical skin forces over a relatively wide area. For instance, the approximate dimensions may be wider than typical wound closure devices such as sutures, glues, tapes (which are typically no more than 1-2 cm wide adjacent to the incision or wound). In various instances, the bonding layer 4 is approximately as wide as the incision is long. This distribution of forces may improve both patient comfort and healing outcome.

The bonding layer 4 may include a variety of features. For instance, in various instances, the bonding layer 4 may include a structural mesh 42. The structural mesh 42 may provide a substrate for other aspects of the wound closure system 2 and may be joinable to a tissue adjacent a wound or incision. The structural mesh 42 may comprise any flexible biocompatible material such as, but not limited to, polyesters, polylactic acid, polypropylene, and nylon. In various embodiments, the bonding layer 4 may include an adhesive tissue interface 41. The adhesive tissue interface 41 may be an adhesive such as a glue. The glue may be cyanoacrylate. The glue may be epoxy or any other glue as desired.

The closure layer 6 may include a first tape member 61 and a second tape member 62. The tape members may be supporting structures of a selective joining member 63 such as a zipper. For instance, the selective joining member 63 may have two sides that are at least partially spaced apart in an open configuration, and that are connected together in a closed configuration. The two sides may be adjacent to an incision or wound and may hold the incision or wound in a healing relation when in the closed configuration. To provide structural support and transfer force from the selective joining member 63, and specifically, the two sides thereof, a corresponding first tape member 61 may be associated with a first side of the selective joining member 63 and a corresponding second tape member 62 may be associated with a second side of the selective joining member 63. In various embodiments, the first tape member 61 and the second tape member 62 may be a mesh material, or a fabric or other textile material, or a plastic material, or a polymeric material, or any desired material.

The closure layer 6 may include a selective joining member 63. The selective joining member 63 may be a zipper as discussed. In various embodiments, the zipper may have specific features. The zipper (and other aspects of the wound closure system 2) may be non-ferrous. For instance, the zipper (and other aspects of the wound closure system 2) may be non-ferrous metal, or plastic, or another material. The zipper (and other aspects of the wound closure system 2) may be any MRI-safe (magnetic resonance imaging safe) material. This facilitates post-procedure patient testing, should a patent need medical imaging during or after a procedure. In various embodiments, the selective joining member 63 may be small hook-like barbs or eyelets on either side (similar to the two sides of a zipper) such as in a shoe, so that a suture or “string-type” device could be “laced-up” and either tied or be glued over.

For instance, a selective joining member 63 may have a first element row 64 and a second element row 65. The first element row 64 may be connected to a first tape member 61 and the second element row 65 may be connected to the second tape member 62. The first element row 64 may be a row of zipper teeth. The second element row 65 may be a row of zipper teeth. The two rows of zipper teeth may selectively connect together or disconnect depending on whether the zipper is open (unzipped) or closed (zipped), respectively.

The selective joining member 63 may have a zipper slider 66. The zipper slider 66 comprises a device that moves along a length of the first element row 64 and second element row 65 moving the rows from unzipped to zipped relation, or from zipped to unzipped relation. To move the zipper slider 66, a user may pull a slider puller 67. A slider puller 67 may comprise a tab attached to the zipper slider 66. The tab may be attached in movable relation to the zipper slider 66, such as by a hinge, and may have an aperture 69 therein. The aperture 69 may facilitate grasping and/or interaction with a closure securement device 8.

The selective joining member 63 may have a bottom stop 68. The bottom stop 68 may comprise a permanent fixation of the first element row 64 to the second element row 65, and/or the first tape member 61 to the second tape member 62 adjacent to an end of the selective joining member 63. The zipper slider 66 may be said to be in a fully unzipped position when impinging against the bottom stop 68.

The wound closure system 2 may include a closure securement device 8. The closure securement device 8 may lock the closure layer 6 in a closed orientation, such as by preventing movement of a zipper slider 66 of zipper. Thus, with reference to both FIGS. 1A-1C and also FIGS. 2A-2C, a closure securement device 8 may include a slider capture boss 81. The slider capture boss 81 may comprise a boss attached to the closure layer 6 at an opposite end from the bottom stop 68. Thus, the zipper slider 66 may be said to be in a fully zipped position when impinging against the slider capture boss 81.

The slider capture boss 81 may define a slider capture cavity 82. The slider capture cavity 82 may have a shape corresponding to an aspect of the selective joining member 63, such as the zipper slider 66 and/or the slider puller 67, to retain the zipper slider 66 in a fully zipped position. In various instances, the slider capture cavity 82 has a floor 83, and the slider puller 67 is inserted into the slider capture cavity 82 against the floor 83. In various embodiments, one or more slider capture tab 85 comprises bosses cantilevering from the floor 83 and through the aperture 69 of the slider puller 67 to enhance retention of the slider puller 67 in the slider capture cavity 82.

The closure securement device 8 may comprise a slider closure lid 84. The slider closure lid 84 maybe a movable flange of the slider capture boss 81 that bends and snap fits over the slider capture cavity 82 to further restrict release of the slider puller 67 from the slider capture cavity 82.

Further embodiments of a closure securement device 8 are contemplated. For instance, rather than a slider capture boss 81 with slider closure lid 84, in various instances, a slider puller 67 is a pull tab that is removable. Moreover, the aperture 69 may facilitate use of a suture to suture the slider puller 67 and zipper slider 66 in fixed position. Finally, the closure securement device 8 may include tape or glue and the slider puller 67 maybe covered with tape and/or glue to affix the slider puller 67 in position. These and other embodiments are contemplated herein.

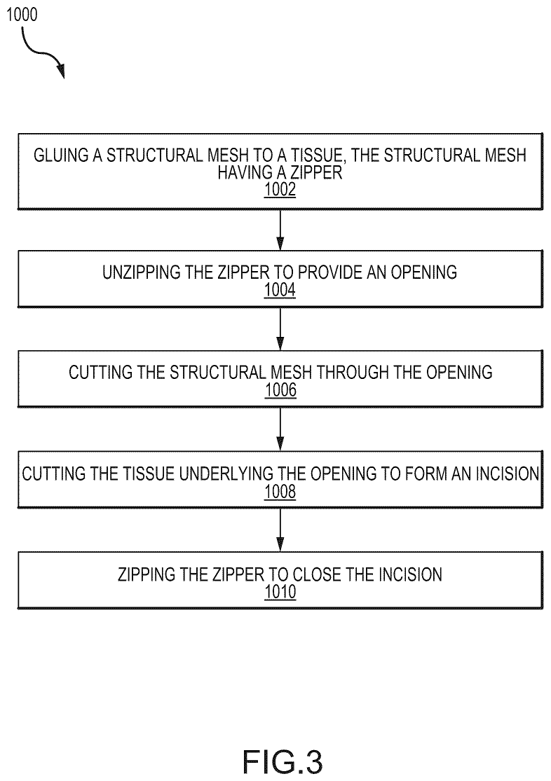

Turning now to FIG. 3, but with continuing reference to FIGS. 1A-2C, an example method of using a wound closure system 2 is provided. The method 1000 may be a method of closing a wound with a wound closure device. The method may include gluing a structural mesh to a tissue (block 1002). The structural mesh may have a zipper attached to a surface of the structural mesh in a first direction. The method may include unzipping the zipper along the first direction to provide an opening in the first direction (block 1004). The method may include cutting the structural mesh through the opening (block 1006). The method may include cutting the tissue underlying the opening to form an incision (block 1008). The method may include zipping the zipper along the first direction to close the incision for healing (block 1010).

Having introduced various devices and a related method, now is a convenient point to provide some example embodiments of the disclosed devices. For example, in various embodiments, a wound closure system 2 is disclosed. The device may have a bonding layer 4, a closure layer 6, and a closure securement device 8. The bonding layer 4 may be adherable to a tissue adjacent a surgical incision site. The closure layer 6 may be lying in a first plane and may be attached to the bonding layer 4. The closure layer 6 may include a selective joining member 63 configured to be selectively closed and opened to selectively define a passage through the closure layer 6. The closure securement device 8 may be attached to at least one of the bonding layer 4 and the closure layer 6 to selectively inhibit transition of the selective joining member 63 from a selectively closed configuration to a selectively opened configuration.

The wound closure system 2 may have one or more additional aspect. For instance, the bonding layer 4 may be a structural mesh 42 adherable to the tissue by glue (e.g., an adhesive tissue interface 41). The selective joining member 63 may be a zipper. The selective joining member 63 may be a zipper having a first element row 64 with a row of teeth and a second element row 65 with a row of teeth disposed adjacent the first element row 64 and selectively engageable to the first element row 64 by a zipper slider 66. The first element row 64 and the second element row 65 may be disposed on opposite sides of the passage in the selectively opened configuration.

In various instances, the closure securement device 8 includes a slider capture boss 81 defining a slider capture cavity 82 conforming in shape to at least a portion of the selective joining member 63 to retain the selective joining member 63 in a fixed position. In various instances, the closure securement device 8 includes a slider capture boss 81 adjacent to an end of the first element row 64 and defining a slider capture cavity 82 shaped to receive the zipper slider 66 in snap-fittable relation. In various instances, the closure securement device 8 includes a slider closure lid 84 attachable to the slider capture boss 81 to restrict removal of the zipper slider 66 from the slider capture cavity 82. The first plane may include one or more complex curvature.

In various instances, the bonding layer 4 is lying in a second plane, and the first plane and the second plane are parallel. Moreover, in various instances the passage is through the closure layer 6 and the bonding layer 4. The passage may be through the closure layer 6 and not through the bonding layer 4. The passage may be through the closure layer 6 and not through the bonding layer 4, and the passage may expose the bonding layer 4 for cutting by a cutting instrument introduced through the passage in the selectively opened configuration.

The selective joining member 63 may include a slider puller 67 attached to the zipper slider 66. The closure securement device 8 may include a slider capture boss 81 defining a slider capture cavity 82 conforming in shape to at least a portion of the slider puller 67 to retain the selective joining member 63 in a fixed position by fixing the slider puller 67 in the slider capture cavity 82. The at least one of the slider capture tab 85 may include a boss extending from a floor 83 of the slider capture cavity 82 to penetrate an aperture 69 of the slider puller 67 to restrict translation of the zipper slider 66 along the first element row 64.

In various instances, the closure layer 6 includes a first tape member 61 supporting the first element row 64 and attached to the structural mesh 42 of the bonding layer 4. The closure layer 6 may include a second tape member 62 supporting the second element row 65 and attached to the structural mesh 42 of the bonding layer 4. The bonding layer 4 may be cuttable along a path adjacent to and disposed between both the first tape member 61 and the second tape member 62 to allow selective opening of the passage. The passage may coincide with the path.

In various instances, the closure layer 6 has a first tape member 61 supporting the first element row 64 and attached to the structural mesh 42 of the bonding layer 4. The closure layer 6 may also have a second tape member 62 supporting the second element row 65 and attached to the structural mesh 42 of the bonding layer 4. The first element row 64 and the second element row 65 may be selectively engageable to each other to selectively close a passage between the first element row 64 and the second element row 65.

Another example wound closure system 2 is provided. The wound closure system 2 may include a structural mesh 42 gluable to a tissue. The wound closure system 2 may include a zipper (selective joining member 63) attached to a surface of the structural mesh 42 in a first direction. The zipper may be selectively openable to provide access to the tissue for cutting. The zipper may be selectively closable to compress the tissue in compression perpendicular to the first direction for healing of an incision at least partially parallel to the first direction.

In some embodiments, the wound closure system 2 has a slider capture boss 81 disposed at an end of the zipper to capture a zipper slider 66 of the zipper to prevent selective opening of the zipper during healing of the incision after the zipper is selectively closed.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching may be used throughout the figures to denote different parts but not necessarily to denote the same or different materials.

Methods, systems, and articles are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

1. A wound closure device comprising:
a bonding layer that is adherable to a tissue adjacent a surgical incision site;
a closure layer lying in a first plane and attached to the bonding layer and comprising a selective joining member configured to be selectively closed and opened to selectively define a passage through the closure layer; and
a closure securement device attached to at least one of the bonding layer and the closure layer to selectively inhibit transition of the selective joining member from a selectively closed configuration to a selectively opened configuration.

2. The wound closure device according to claim 1, wherein the bonding layer is a structural mesh that is adherable to the tissue by glue.

3. The wound closure device according to claim 1, wherein the selective joining member comprises a zipper.

4. The wound closure device according to claim 1, wherein the selective joining member comprises a zipper having a first element row comprising a row of teeth and a second element row comprising a row of teeth disposed adjacent the first element row and selectively engageable to the first element row by a zipper slider.

5. The wound closure device according to claim 4, wherein the first element row and the second element row are disposed on opposite sides of the passage in the selectively opened configuration.

6. The wound closure device of claim 1, wherein the closure securement device comprises a slider capture boss defining a slider capture cavity conforming in shape to at least a portion of the selective joining member to retain the selective joining member in a fixed position.

7. The wound closure device of claim 4, wherein the closure securement device comprises a slider capture boss adjacent to an end of the first element row and defining a slider capture cavity shaped to receive the zipper slider in snap-fittable relation.

8. The wound closure device according to claim 7, wherein the closure securement device comprises a slider closure lid attachable to the slider capture boss to restrict removal of the zipper slider from the slider capture cavity.

9. The wound closure device according to claim 1, wherein the first plane includes one or more complex curvature.

10. The wound closure device according to claim 1,
wherein the bonding layer is lying in a second plane, and
wherein the first plane and the second plane are parallel.

11. The wound closure device according to claim 1, wherein the passage is through the closure layer and the bonding layer.

12. The wound closure device according to claim 1, wherein the passage is through the closure layer and is not through the bonding layer.

13. The wound closure device according to claim 1,
wherein the passage is through the closure layer and is not through the bonding layer, and
wherein the passage exposes the bonding layer for cutting by a cutting instrument introduced through the passage in the selectively opened configuration.

14. The wound closure device according to claim 4,
wherein the selective joining member comprises a slider puller attached to the zipper slider, and
wherein the closure securement device comprises a slider capture boss defining a slider capture cavity conforming in shape to at least a portion of the slider puller to retain the selective joining member in a fixed position by fixing the slider puller in the slider capture cavity.

15. The wound closure device according to claim 14, comprising at least one slider capture tab comprising a boss extending from a floor of the slider capture cavity to penetrate an aperture of the slider puller to restrict translation of the zipper slider along the first element row.

16. The wound closure device according to claim 4,
wherein the closure layer comprises a first tape member supporting the first element row and attached to a structural mesh of the bonding layer,
wherein the closure layer comprises a second tape member supporting the second element row and attached to the structural mesh of the bonding layer,
wherein the bonding layer is cuttable along a path adjacent to and disposed between both the first tape member and the second tape member to allow selective opening of the passage, and
wherein the passage coincides with the path.

17. The wound closure device according to claim 4,
wherein the closure layer comprises a first tape member supporting the first element row and attached to a structural mesh of the bonding layer,
wherein the closure layer comprises a second tape member supporting the second element row and attached to the structural mesh of the bonding layer, and
wherein the first element row and the second element row are selectively engageable to each other to selectively close the passage between the first element row and the second element row.

18. A wound closure device comprising:
a structural mesh that is gluable to a tissue; and
a zipper attached to a surface of the structural mesh in a first direction,
wherein the zipper is selectively openable to provide access to the tissue for cutting, and
wherein the zipper is selectively closable to compress the tissue in compression perpendicular to the first direction for healing of an incision at least partially parallel to the first direction.

19. The wound closure device according to claim 18, further comprising a slider capture boss disposed at an end of the zipper to capture a zipper slider of the zipper to prevent selective opening of the zipper during healing of the incision after the zipper is selectively closed.

20. A method of closing a wound with a wound closure device comprising:
gluing a structural mesh to a tissue, the structural mesh having a zipper attached to a surface of the structural mesh in a first direction;
unzipping the zipper along the first direction to provide an opening in the first direction;
cutting the structural mesh through the opening;
cutting the tissue underlying the opening to form an incision; and
zipping the zipper along the first direction to close the incision for healing.
